(19) United States
(12) Patent Application Publication
(10) Pub. No.: US 2021/0386304 A1
(43) Pub. Date: Dec. 16, 2021

(54) SYSTEMS AND METHODS FOR DETECTING RESPONSE TO NON-INVASIVE ELECTROMAGNETIC SIGNALS

(71) Applicant: 149 Medical, Inc., Worcestor, MA (US)

(72) Inventor: Bernhard Benedikt Zimmermann, Cambridge, MA (US)

(21) Appl. No.: 17/345,793

(22) Filed: Jun. 11, 2021

Related U.S. Application Data
(60) Provisional application No. 63/038,295, filed on Jun. 12, 2020.

(51) Int. Cl. A61B 5/0265; A61B 5/00

(52) U.S. Cl. CPC A61B 5/0265; A61B 5/0075; A61B 2562/0271; A61B 2562/0219; A61B 5/6801

(57) ABSTRACT

Disclosed herein are systems and methods for the continuous monitoring of subjects that ensure that the probe of a monitoring device that needs to be in contact with the subject remains in continuous contact with the subject. Embodiments of the present disclosure include contacting a probe to a subject, transmitting a first electromagnetic radiation signal from a source in the probe to the subject, detecting via at least one detector in the probe a second electromagnetic radiation signal from the subject, detecting via a processor coupled to the at least one detector a change in contact between the probe and the subject based on a change in the second electromagnetic radiation signal, and stopping via the processor the source from transmitting the first electromagnetic signal upon detecting the change in contact between the probe and the subject.

### CROSS REFERENCE

This application claims priority to U.S. provisional application Ser. No. 63/038,295 filed Jun. 12, 2020, the disclosure of which is incorporated herewith in its entirety.

### FIELD

The present disclosure relates to systems and methods for the continuous monitoring of physiological markers.

### BACKGROUND

Detecting changes in oxygen delivery to the brain is critical to monitoring patients with ischemic perfusion injuries, traumatic brain injury, and hemorrhaging. Without adequate detection of changes in oxygen delivery and cerebral blood flow (CBF), patients can endure extended periods without adequate blood supply to the brain, resulting in irreversible damage. Detecting changes in oxygen delivery therefore requires continuous bedside monitoring of CBF to allow for early intervention.

Various non-invasive techniques have been employed for monitoring of CBF. Near-infrared Spectroscopy (NIRS) devices, for example, use a probe with an electromagnetic signal to measure absorption of the electromagnetic signal by cerebral blood. Existing NIRS devices, however, can only measure CBF.

Moreover, continuous monitoring of patients using NIRS-like devices requires maintaining constant communication between the probe and the patient for the entire period of monitoring—often as long as three days. Movement by patients during this period, however, often results in loss of contact between the probe and the patient, resulting in inaccurate readings and a failure to detect and intervene in ischemic events. This problem is only exacerbated in infant patients, where the size and rigidity of NIRS devices results in discomfort for the patient, resulting in patient movement. As a result, despite improvements in suitable interventions for ischemic events, the inability to effectively continuously monitor patients has resulted in a significant fatality rate among patients at risk for ischemic events, especially infant patients.

### SUMMARY

The present disclosure provides systems and methods for the continuous monitoring of subjects that ensure that the probe of a monitoring device that needs to be in contact with the subject remains in continuous contact with the subject.

Systems of the present disclosure determine contact between the probe and the subject using the probe itself. The probe includes a source configured to transmit a first electromagnetic radiation signal to the subject and at least one detector configured to detect a second electromagnetic radiation signal from the subject. A processor coupled to the at least one detector is provided instructions by non-transitory memory and executes those instructions to detect a change in contact between the probe and the subject based on a change in the second electromagnetic radiation signal. The processor can then cause the system to stop the source from transmitting the first electromagnetic signal and alert a clinician of the loss of contact. The clinician may then reposition the probe, preventing an otherwise extended period with a loss of contact between the probe and the patient that could result in a fatal failure to detect and intervene in an ischemic event. This will also provide for additional safety for the patient and clinical personnel.

Methods of the present disclosure may be performed by using the systems of the present disclosure. Methods for monitoring contact between a probe and a subject of the present disclosure comprise contacting a probe to a subject, transmitting a first electromagnetic radiation signal from a source in the probe to the subject, detecting via at least one detector in the probe a second electromagnetic radiation signal from the subject, detecting via a processor coupled to the at least one detector a change in contact between the probe and the subject based on a change in the second electromagnetic radiation signal, and stopping via the processor the source from transmitting the first electromagnetic signal upon detecting the change in contact between the probe and the subject.

The present disclosure can be used with various animal subjects that may be prone to movement, including mammalian subjects and human subjects. Advantageously, the present invention may be used with neonates and infants, for example infants less than 25 weeks of age.

The present disclosure may also be used together with any known monitoring device that requires communication between the device and the subject. For example, devices and methods of the present disclosure may be used together with a Near-Infrared Spectroscope (NIRS), diffuse correlative spectroscope (DCS), cerebral oximeters, or Laser speckle contrast imaging (LSCI) device. Accordingly, the first electromagnetic signal transmitted by the probe may be a near-infrared signal.

Systems and methods of the device detect contact between the probe and the subject based on a change in a second electromagnetic radiation signal from the subject and, accordingly, the change in intensity, a change in wavelength, and/or a Doppler shift of the second signal. Advantageously, systems and methods of the device detect changes in contact between the probe and the subject in real time, immediately detecting a loss of contact between the probe and the subject.

Systems and methods of the device may further comprise a wearable article configured to house the probe and maintain contact between the probe and the subject. For example, the wearable article may be a cap fitted over the top of the head of the subject and/or a Continuous positive airway pressure (CPAP) machine.

Systems and methods of the present disclosure can be non-invasive and contact between the probe and the subject may be contact between the probe and a tissue of the subject, for example the skin of the subject.

System and methods of the present disclosure may further comprise an accelerometer or temperature sensor to detect a change in contact between a probe and a subject. The accelerometer or temperature sensor can be used together with any of the system described above. In other aspects of the present disclosure, an accelerometer and/or temperature sensor is used to detect the change in contact between a probe and the subject without the use of a detector or second electromagnetic signal.

For example, systems and methods of the present disclosure may monitor contact between a probe and a subject using the system comprising a probe configured to contact a subject, the probe including a source configured to transmit a first electromagnetic radiation signal to the subject; and at least one of an accelerometer or temperature sensor. The system further comprises a processor coupled to the at least one detector, and non-transitory memory containing instructions executable by the processor to cause the system to detect a change in contact between the probe and the subject based on a change detected by the accelerometer or temperature sensor; and stop the source from transmitting the first electromagnetic signal. Advantageously, the system may comprise both an accelerometer and a temperature sensor. Where both an accelerometer and temperature sensor are used, the non-transitory memory may contain instructions executable by the processor to cause the system to detect a change in contact between the probe and the subject based on a change detected by both the accelerometer and temperature sensor.

### BRIEF DESCRIPTION OF THE DRAWINGS

FIG. 1 shows a measurement device that transmits an electromagnetic radiation signal to detect a speckle pattern from scattering of the signal by red blood cells.

FIG. 2 shows a probe according to some aspects of the present disclosure.

FIG. 3 shows a probe according to some aspects of the present disclosure.

FIG. 4 shows a cross-section of a probe with two detectors, a single source, and an accelerometer according to some aspects of the present disclosure.

FIG. 5 shows a probe with two detectors, a single source, and an accelerometer according to some aspects of the present disclosure.

FIG. 6 shows a diffuser and prism according to some aspects of the present disclosure.

FIG. 7 shows a source near a detector housing without a diffuser according to some aspects of the present disclosure.

FIG. 8 shows a source near a detector housing with a diffuser according to some aspects of the present disclosure.

FIG. 9 shows a source near a detector housing according to some aspects of the present disclosure.

FIG. 10 shows a detector with an accelerometer and a mirror according to some aspects of the present disclosure.

FIG. 11 shows a detailed view of an accelerometer that can be used according to some aspects of the present disclosure.

FIG. 12 shows a schematic representation of a system according to some aspects of the present disclosure.

FIG. 13 shows a blood flow monitor that can be used in some aspects of the present disclosure.

FIG. 14 shows a system of the present disclosure used in connection with a wearable article according to some aspects of the present disclosure.

FIG. 15 shows another view of the system of FIG. 14.

FIG. 16 shows another view of the system of FIG. 14

FIG. 17 shows fiber cable that can be used with probes according to some aspects of the present disclosure.

FIG. 18 shows packaging for fiber cables that can be used to protect and transport fiber cables.

### DETAILED DESCRIPTION

Systems of the present disclosure determine contact between the probe of a monitoring device and a subject. The present disclosure may also be used together with any known monitoring device that requires communication between the device and the subject. For example, the present disclosure may be used together with a Near-Infrared Spectroscope (NIRS), diffuse correlative spectroscope (DCS), cerebral oximeters, a Laser speckle contrast imaging (LSCI) device, or a device for measuring hemoglobin oxygenation. Examples of NIRS devices may be devices as described in Hori et al. (2015) Anesth Analg. 2015 November; 121(5): 1187-1193. Examples of LSCI devices may be as described in Lu et al. (2015) Conf Proc IEEE Eng Med Biol Soc. 2015 August; 2015: 6971-6974. Examples of DCS devices may be as described in Shang et al. (2013) “Clinical applications of near-infrared diffuse correlations spectroscopy and tomography for tissue blood flow monitoring and imaging”, Anat. Physiol. 3(2):128; or Kim et al. (2010) “Noninvasive Measurement of Cerebral Blood Flow and Blood Oxygenation Using Near-Infrared and Diffuse Correlation Spectroscopies in Critically Brain-Injured Adults”, Neurocrit Care 12(2):173-180, each of the above references which are incorporated by reference.

Accordingly, the first electromagnetic signal transmitted by the probe may be a near-infrared signal. The source may be a laser. Advantageously, the source may be about 700 to about 900 nm, about 785 nm, or about 852 nm. For example, the source may be a 785 nm laser as provided by TOPTICA Photonics Inc., Farmington, N.Y., for example a Topica IBEAM-SMART-785-S-WS laser. The source may be a laser as provided by CrystaLaser, Reno, Nev., for example a DL852-100-SO.

Lasers may be long coherence length lasers. Lasers may have an output power of about 100 mW. Lasers advantageously may be designed to stay below the American National Standards Institute (ANSI) standards. For example, probes of the present disclosure may comprise a source transmitting a first electromagnetic signal with about a 3.5 mm diameter and about a 785 nm limit and the maximum about 30 mW limit may be set to about 26 mW maximum. For example, an 850 nm first electromagnetic signal a maximum of about 40 mW limit may be set to 35 mW. For a signal with a maximum of about 25 mW the limit may be set to 15 mW.

Continuous monitoring may be for extended periods, for example about one day, about two days, about three days, or greater than three days. During the period of continuous monitoring, the probe may also be repositioned to a different position in contact with the subject, for example the probe may be moved approximately every 6-8 hours. In methods of the present disclosure, a clinician may periodically verify that no skin irritation has developed from the probe.

Systems and methods of the device detect contact between the probe and the subject based on a change in a second electromagnetic radiation signal from the subject. Accordingly, the change in the second electromagnetic signal may be measured directly from the physiological marker being monitored by the device or may be measured from a different physiological marker. For example, the change may be selected from the group consisting of a change in intensity, a change in wavelength, and a Doppler shift of the second signal.

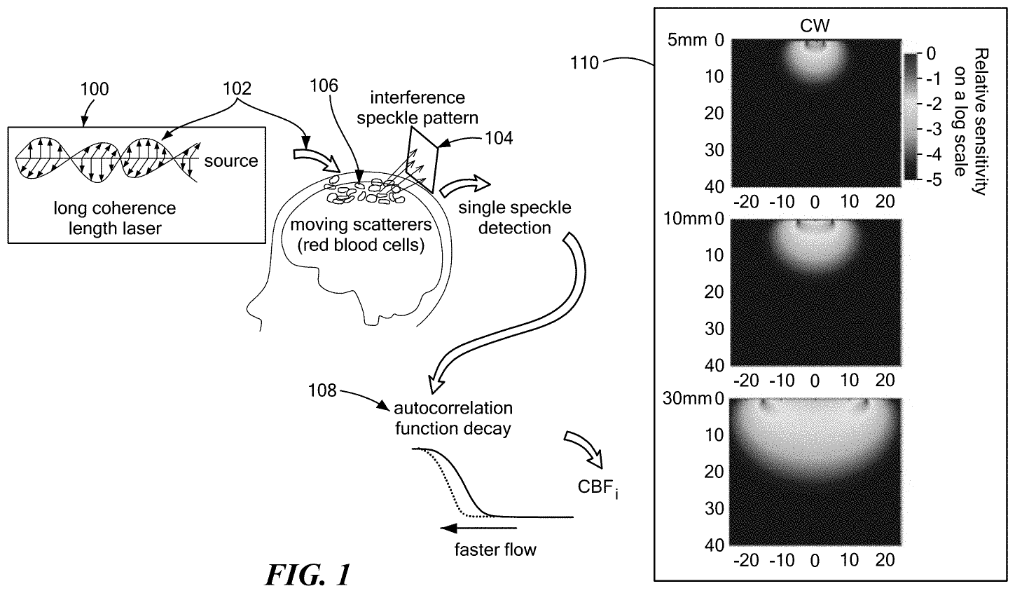

FIG. 1 shows a measurement device or probe 100 that transmits an electromagnetic radiation signal 102 to detect a speckle pattern 104 from scattering of the signal by red blood cells 106.

The fluctuating speckle pattern 104 represents a Doppler shift that can be analyzed by calculating an autocorrelation function 108 of the intensity decay over time. This correlation can then be fit to obtain a cerebral blow flow index 110 (CFBi) giving an indication of CFB.

Probes of the present disclosure include a source configured to transmit a first electromagnetic radiation signal to the subject and at least one detector configured to detect a second electromagnetic radiation signal from the subject. Probes may be lightweight probes or may be heavier probes. Probes may advantageously be sanitized before use. Probes may also be single use, multi-use or disposable.

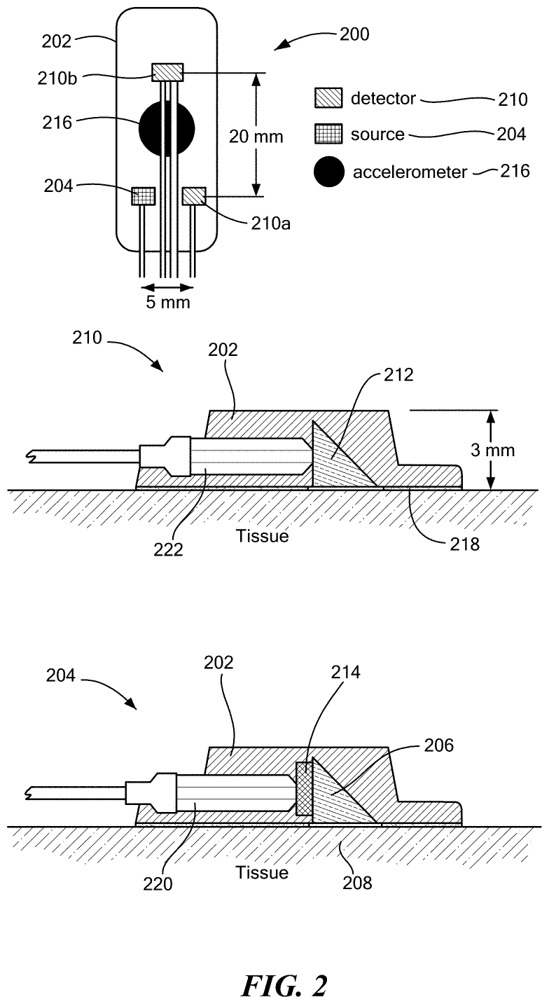

FIG. 2 depicts a portion of a probe 200 according to aspects of the present disclosure. The probe comprises a housing 202 with contains a source 204 which transmits an electromagnetic radiation signal to a mirror or prism 206 with redirects the signal towards the subject 208.

Probes of the present invention comprise at least one detector 210, for example 1, 2, 3, 4, 5, 6, 7, 8, or greater than 8 detectors. In various probes 200 the electromagnetic signal travels from the source 204 through part of the subject 208, such as a target tissue, and back to the one or more detectors 210. The separation distance between the source 204 and detector 210, 210a, 210b impacts the depth into the tissue 208 that is measured. Accordingly, the distance between the source 204 and the one or more detectors 210 may be adjusted accordingly, for example with a first short detector 210a placed proximate to the source 204 and a second long detector 210b placed further from the source. In some embodiments, the first detector 210a may be between about 3 mm to about 8 mm from the source 204 and the second detector 210b may be about 20 mm to about 25 mm from the source 204. Advantageously, the difference between two signals can be calculated for more sensitive readouts of physiological activity. By way of non-limiting example, the detectors 210, 210a, 210b may be single photon detectors as sold by Hamamatsu Photonics K.K., Naka-ku, Hamamatsu City, Shizuoka, Japan, for example a Hamamatsu detector model C14463- 05GD. Single photon detectors may output about a 5V pulse for every detected photon.

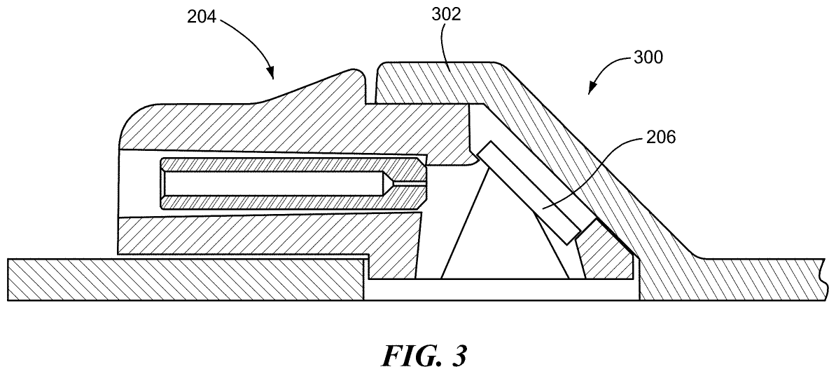

FIG. 3 depicts a portion of a probe 300 comprising a source portion 204 as depicted in FIG. 2 and further comprising a short detector and long detector housings (not shown) within the probe.

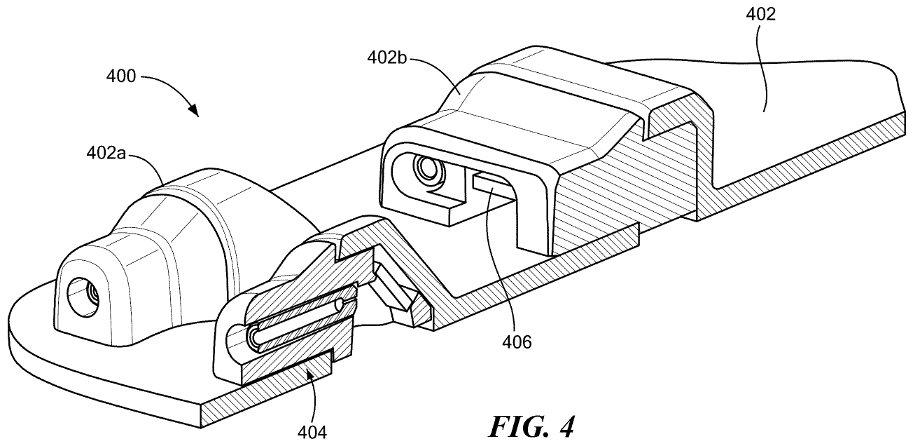

FIG. 4 depicts a cross-section of a probe 400 with two detectors 402a, 402b and a single source 404, with the short detector 402a closer to the source 404 and the long detector 402b positioned further from the source 402. Advantageously, the probe further 400 comprises an accelerometer 406 pictured near the long detector 402b. The accelerometer 406 can be a printed circuit board that can be coupled to a housing 408 of the probe 400 at a location that aligns with or is near to the long detector 402b.

Examples of accelerometers that may be used with the present invention may be those provided by STMicroelectronics N.V., headquartered in Geneva, Switzerland, such as the LIS2DW12, or may be those provided by Bosch Sensortec GmbH, headquartered in Reutlingen, Germany, such as the BMI160. Advantageously, the accelerometer may comprise an integrated temperature sensor, for example as integrated into the LIS2DW12 accelerometer or BMI160. Advantageously, the accelerometer may also comprise an integrated gyroscope, for example as integrated into the BMI160.

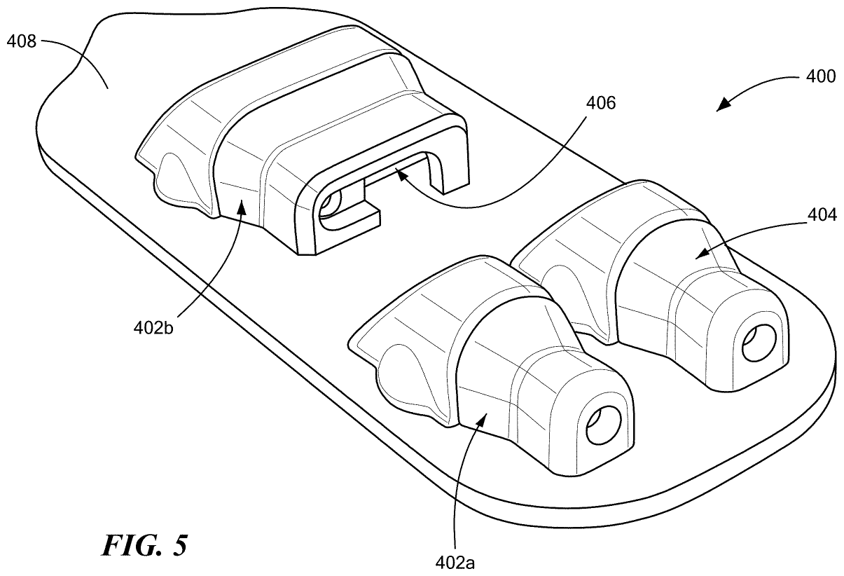

FIG. 5 shows another view of the probe 400 depicted in FIG. 4 with two detectors 402a, 402b, a single source 404, and an accelerometer 406 each pictured.

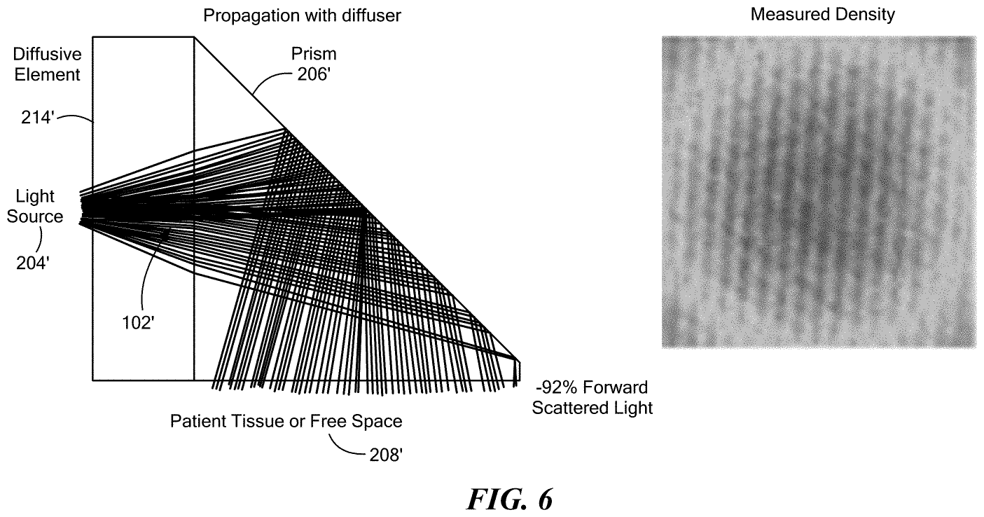

Probes of the present disclosure may also comprise one or more prisms or mirrors 206 (see FIG. 2 and FIG. 6) to redirect the first electromagnetic signal 102, 102′ from the source 204 to the tissue 208, 208′ of the subject. For example, the prism 206 may redirect the first electromagnetic signal 102, 102′, e.g., in the form of light, about 90 degrees towards the patient's tissue 208, 208′. This may be advantageous as it allows the source 204 to first transmit the first electromagnetic signal 102, 102′ parallel or substantially parallel with respect to the subject's tissue 208, 208′ and thereafter be redirected to the subject's tissue. This allows probes of the present disclosure to be compact where the source 204 may have a length that would otherwise prevent the probe from being placed under the subject's clothing, for example under a cap. Prisms 206 used in connection with the source 204 may advantageously be placed at the end of the probe further from a subject's eyes to prevent discomfort and additional safety.

Prisms 212 (see FIG. 2) may also be used to redirect the second electromagnetic signal towards the one or more detectors 210a, 210b. Prisms advantageously may have a coating that increases transmission and reflection over desired wavelength ranges. For example, a silver coating or an aluminum coating may be used. When used to redirect the second electromagnetic signal towards the one or more detectors 210a, 210b, transmission efficiency may be of particular importance, as probes may lose about 20 to about 30% efficiency or greater than 30% efficiency. For example, this may result in detectors having at least 60% efficiency for each channel and/or an average of at least 80% efficiencies across all channels. The signal source may operate between about 40% and about 60% efficiency when used together with a diffusor. A stamped metal piece or plastic mirror may be used in place of a prism.

Probes of the present disclosure may also comprise one or more diffusers 214 (see FIG. 2) for propagating the first electromagnetic signal transmitted from the source 204 over an increased area, for example over a greater section of a subject tissue 208. For example, the diffuser 214 may be a holographic diffuser or Teflon diffuser used to spread the first electromagnetic signal. The diffuser 214 may propagate the signal over about a 3.5 mm by 3.5 mm area. Prisms 206 may be used to redirect the first electromagnetic signal after propagation by a diffuser 214. For example, where the diffuser 214 propagates the signal over about a 3.5 mm by 3.5 mm area, about a 3.5 mm by 3.5 mm source prism 206 may work together with the diffuser to redirect the propagated signal to the subject.

FIG. 6 depicts a probe according to aspects of the present disclosure with a source 204′ together with a diffusive element 214′ which propagates the first electromagnetic radiation signal 102′ and a prism 206′ which redirects the signal approximately 90 degrees. The diffuser 214′ has a transmission efficiency of about 92% of forward scattered light. Probes, prisms, and diffusive elements that may be used together with the present disclosure include those described in U.S. Patent Application Publication No. 2017/0027447, filed on Oct. 11, 2016 and entitled “System and Method for Improved Light Delivery to and from Subjects,” the entirety of which is incorporated herein.

Advantageously, as shown in FIGS. 2-5, the probe housing 202, 302, 402 may be black to prevent ambient light from entering the area of the subject to which the first electromagnetic signal 102′ is directed. Holes (not shown) may further be cut into the housing 202, 302, 403 to make the probe more flexible, for example to contour to the area of contact between the probe and the subject.

Probes may be designed combining any of the above described features in combination.

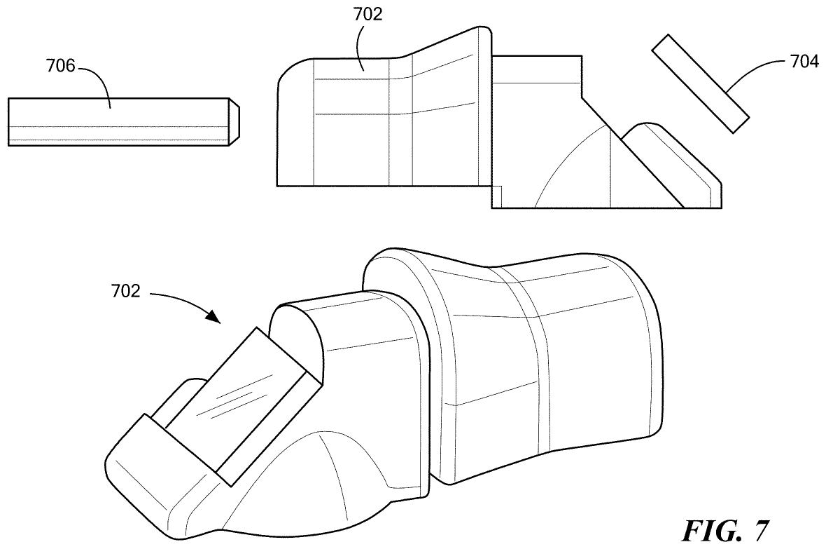

FIG. 7, for example, depicts a probe comprising a source 702 near a detector (not shown) with a mirror 704 without use of a diffuser.

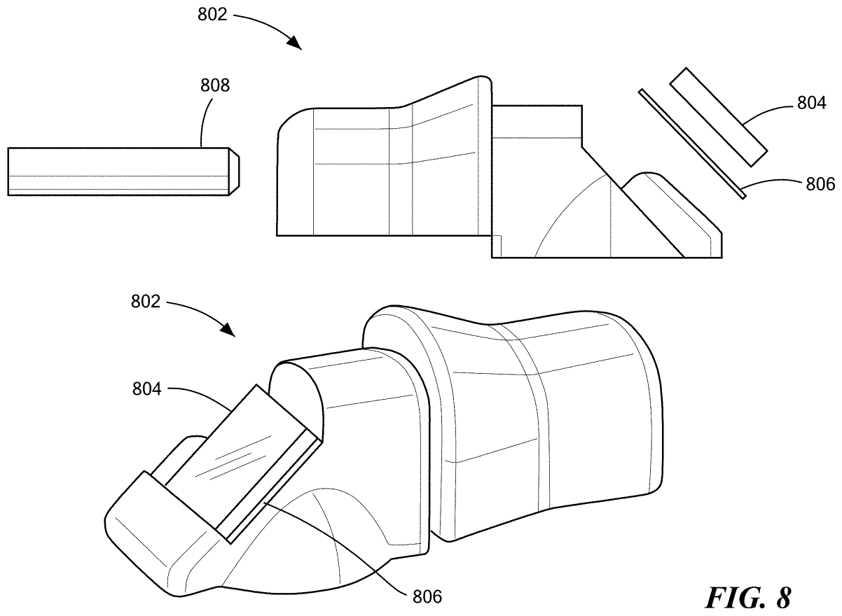

FIG. 8, alternatively, depicts a probe comprising a source 802 near a detector (not shown) with a mirror 804 and a diffuser 806.

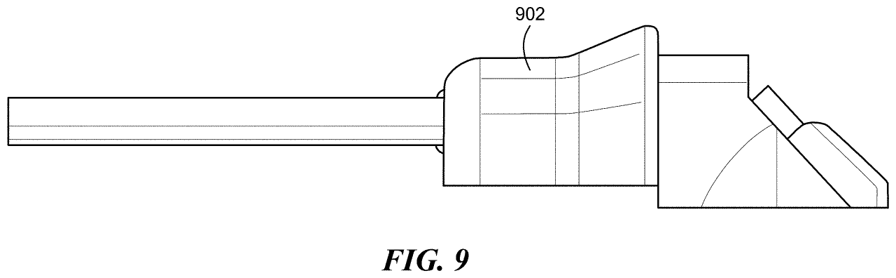

FIG. 9 depicts a source 902 near a detector (not shown) generally according to some aspects of the present disclosure.

The present disclosure can be used with various animal subjects that may be prone to movement, including mammalian subjects and human subjects. Advantageously, the present disclosure may be used with neonates and infants, for example infants under about 1 kg (about 1 to about 2 pounds), and/or less than about 25 weeks of age and/or with about a head circumference less than about 28 cm.

Accordingly, probes of the present disclosure may also comprise an accelerometer 216, 406, to sense movements by the subject. For example, the movement detected may be head movement by the subject. The accelerometer 214, 406 may advantageously be placed near the back of the probe. Detection of head movement by the accelerometer may be used to discard readings by the detectors in order to distinguish real signals from signals caused by the movement rather than by a decrease in contact between the probe and the subject.

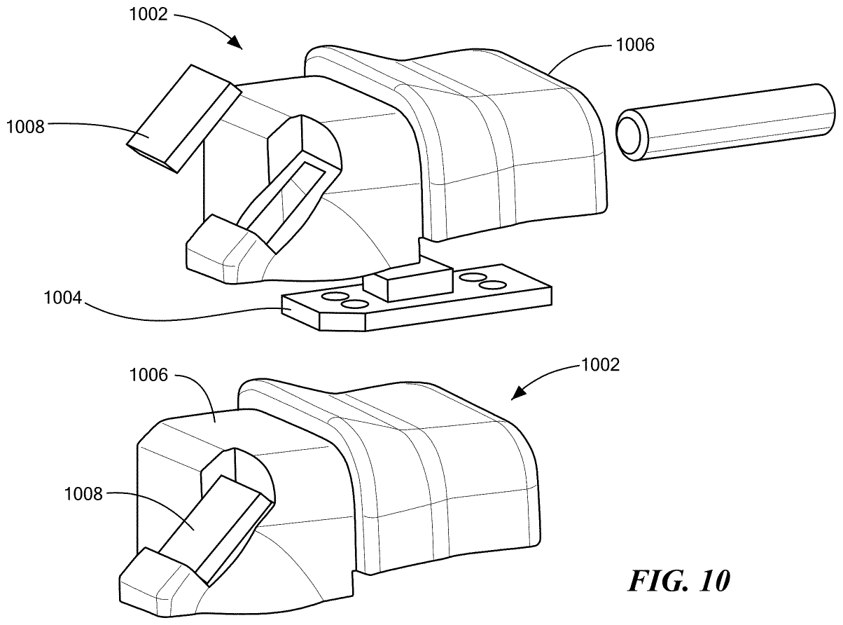

FIG. 10 shows a probe with a detector 1002 and an accelerometer 1004 proximate to the detector aspects of the present disclosure. Detector aspects can include, for example, a housing 1006 and/or a mirror 1008 or prism of the detector 1002.

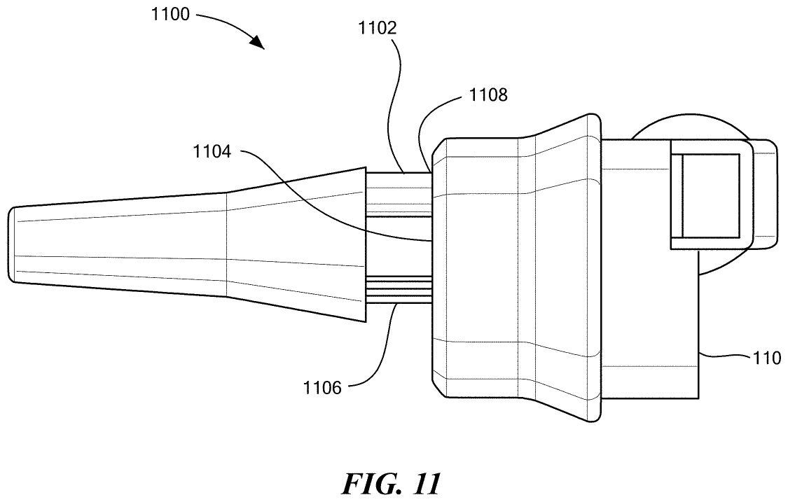

FIG. 11 shows a detailed view of an accelerometer 1100 that be used according to some aspects of the present disclosure. The accelerometer 1100 can include, among other things, a strain relief 1102, a sheathing 1104, electrical wires 1106, and single mode fibers 1108. The sheathing 1104 can abut or otherwise be in contact with or coupled to a detector housing 1110.

Systems and methods of the present disclosure can use a processor coupled to the at least one detector is provided instructions by non-transitory memory and executes those instructions to detect a change in contact between the probe and the subject based on a change in the second electromagnetic radiation signal. The processor can then cause the system to stop the source from transmitting the first electromagnetic signal and alert a clinician of the loss of contact. The clinician may then reposition the probe, preventing an otherwise extended period with a loss of contact between the probe and the patient that could result in a fatal failure to detect and intervene in an ischemic event. Advantageously, systems and methods of the device detect changes in contact between the probe and the subject in real time, immediately detecting a loss of contact between the probe and the subject.

Systems and methods of the present invention may also comprise a controller for user input. Systems may include a medical 12V supply, a power distribution board, a power switch, and LEDs indicating whether the source is transmitting an electromagnetic signal. The controller and processor may be housed in a single chassis or in separate chassis and may include a fan for cooling the components. The controller and/or processor may be housed in a laptop. The laptop may comprise a Windows Application to be used by the controller and/or processor.

Measurements of the second electromagnetic radiation signal from the one or more detectors may be sent to the processor and changes in contact between the probe and the subject based on a change in the second electromagnetic radiation signal may be calculated, for example from correlations calculated by the processor. The process may further determine a CBFi. The processor may send an alert to the controller, for example a laptop, indicating that the probe is not in contact with the subject. The processor may stop the source from transmitting the first electromagnetic signal. Advantageously, the processor may be coupled to the power distribution board or power switch to prevent power from reaching the source in order to stop the source from transmitting the first electromagnetic signal.

The processor may also send an alert to the controller if any component of the system is not connected. The controller and processor may each comprise a field-programmable gate array (FPGA), for example an Intel® FPGA as sold by Intel, Santa Clara, Calif. The FGPA may calculate correlations between the second electromagnetic signal detected by the detector and connectivity between the probe and the subject. The FGPA may calculate correlations between the second electromagnetic signal and the physiological markers being measured by the measurement device.

The system may further comprise a power monitor to monitor output power from the source. For example, 1% of the first electromagnetic signal from the source may be transmitted to the power monitor. The processor may be provided instructions from the non-transitory memory to stop the source from the transmitting the first electromagnetic signal in response to a change in power detected by the power monitor. Alternatively, the controller may be used by used to stop the source from transmitting the first electromagnetic signal. The system may further comprise a signal quality monitor. In methods of the present invention, the processor may provide an alert if a low signal quality is indicated or a clinician may periodically monitor the signal quality.

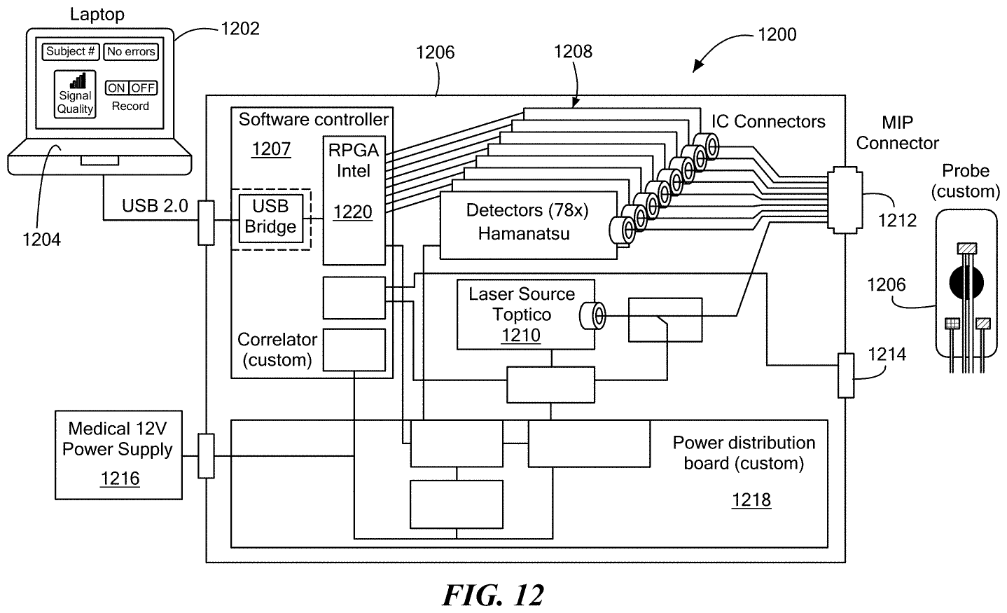

FIG. 12 depicts a schematic representation of a system 1200 according to some aspects of the present disclosure. The system comprises a laptop 1202 that can include a controller for user input, e.g., a keyboard 1204, mouse, etc. Optionally, the laptop may house the processor for the system. The laptop is coupled to the probe 1206 with a standard USB 2.0 connector 1208. It will be appreciated that other types of electrical connections, including wired or wireless connections, can be made between the laptop 1202 and the probe 1206. The laptop 1202 together with the controller allows for control of the system 1200 through software 1207. The probe 1206 depicted comprises 7 to 8 detectors 1208 and a single source 1210. The detectors 1208 are each coupled to the system using an MTP® connector 1212, as sold by US Conec Ltd., Hickory, N.C. Alternative connectors that can provide the same or similar connective functionality may be used. The system further comprises an accelerometer 1214. The system is powered by a power supply 1216, such as a medical 12V power supply, coupled to a power distribution board 1218. The system 1200 includes a field-programmable gate array (FPGA) that may be used by the processor and controller.

Systems and methods of the device may further include a wearable article configured to house a probe of the present disclosure and maintain contact between the probe and the subject. For example, the wearable article may be a cap fitted over the top of the head of the subject and/or a Continuous positive airway pressure (CPAP) machine. In some methods the present disclosure the cap is a knit cap and the CPAP machine is together with the cap. The cap may already be in place and the probe is applied by lifting up the cap and sliding the probe under the cap to secure the cap in place. In some methods of the present disclosure, the probe is placed such that it does not interfere with the foam or straps of the CPAP mask and any wires are directed away from the face of the subject. For example, wires may be taped to the top of the subject's head to keep them out of the way of the subject's face.

Systems and methods of the present disclosure can be non-invasive and contact between a probe and the subject may be contact between the probe and a tissue of the subject, for example the skin of the subject. The probe may comprise a hydrogel 218 (see FIG. 2) at the point of contact between the probe and the subject, for example, an AmGel® hydrogel as sold by Axelgaard Manufacturing Co. Ltd., Fallbrook, Calif. Advantageously, a moisturizer may be used together with the hydrogel, for example when used with a subject with dry skin. The hydrogel may also contain a cut-out designed in relation to the probe. The hydrogel can also be disposable and can be replaced on the probe.

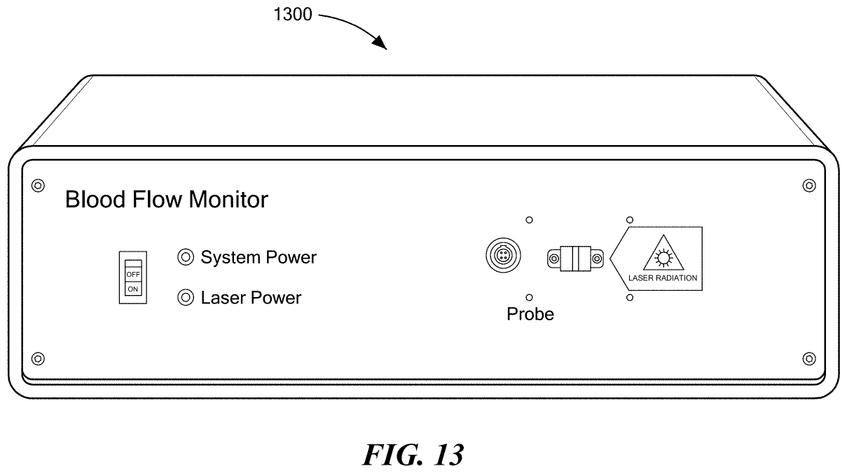

FIG. 13 shows a blood flow monitor 1300 that can be used in some aspects of the present disclosure, for example in the system of FIG. 12. As pictured, various non-invasive techniques have been employed for monitoring of CBF. Near-infrared Spectroscopy (NIRS) devices, for example, use a probe with an electromagnetic signal to measure absorption of the electromagnetic signal by cerebral blood. Existing NIRS devices, however, cannot measure CBF.

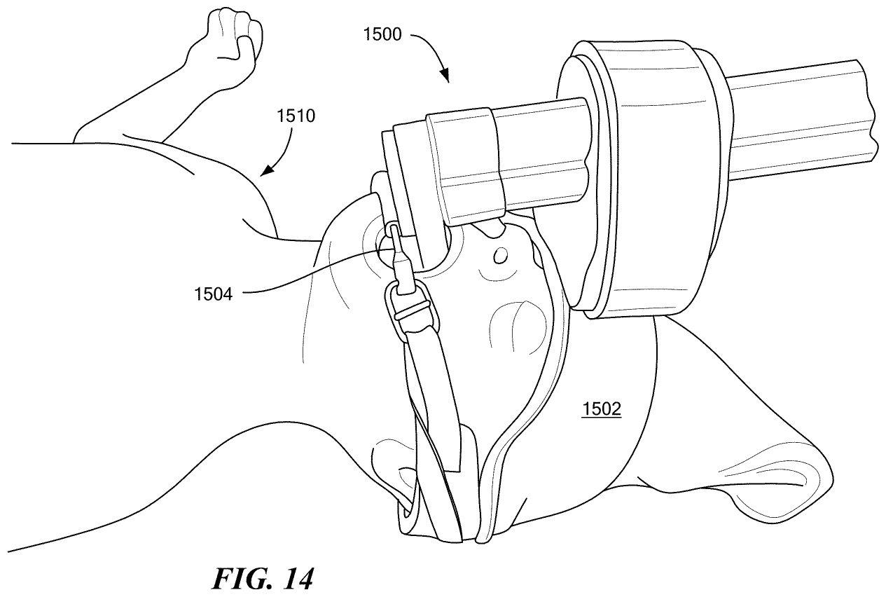

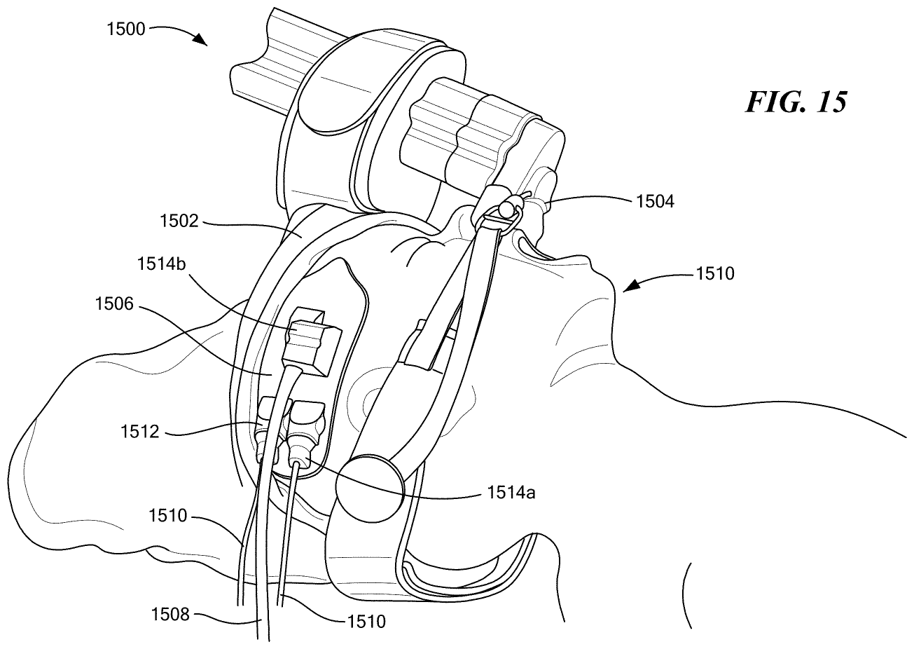

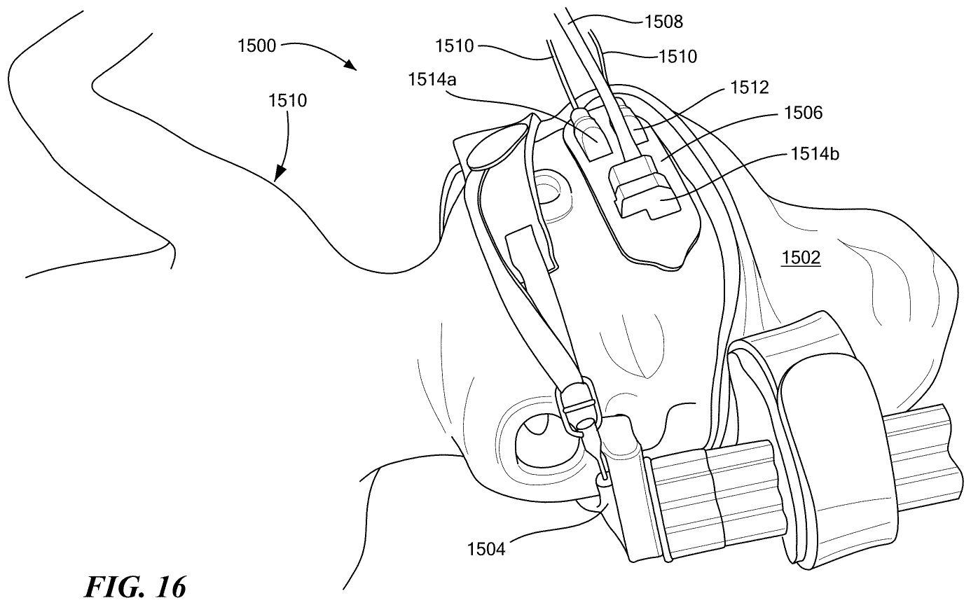

FIG. 14-16 depict one embodiment of a system 1500 of the present disclosure used in connection with a knit cap 1502 and CPAP mask 1504 according to some aspects of the present disclosure. A probe 1506 in accordance with the present disclosure is positioned under the knit cap 1502 and communicably coupled to a processor, e.g., the laptop 1202 (see FIG. 12) via a fiber cable 1508 such that loss of contact between the probe and the subject 1510 is reported to a clinician and a source of the probe 1506 is stopped from transmitting the first electromagnetic signal.

One or more fibers 1508, 1510 may be used to couple components of the probe 1506. Advantageously, fibers 1508, 1510 may be very flexible. The fiber 1508, 1510 may be a braided cable. Fibers 1508, 1510 may be used to couple a source 1512 and detectors 1514a, 1514b and may advantageously be configured to minimize crosstalk between source and detector fibers, for example using black coatings on the fibers. Fibers may be single mode fibers optimized for specific wavelengths, for example about 780 nm to about 970 nm. Fibers may be 780HP fibers as sold by Nufern, East Granby, Conn. or OEM fibers as sold by Thorlabs, Newton, N.J. Single mode fibers may also be selected to cover optical bands, for example about 980 mn to about 1650 nm and/or may be selected based on diameter, for example about 9 μms, however such fibers may provide a greater first signal at the expense of worse signal quality. Multi-mode fibers may also be used, for example multi-mode fibers with about a 125 μms outer diameter. Advantageously, smaller diameter fibers may be used to work with various connectors. A mix of fibers can be used together. For example, the source 1512 may be coupled to a diffuser 214 or prism 206 (see FIG. 2) with a multi-mode fiber. The short detector 1514a and/or long detector 5114b may be coupled to a prism 212 (see FIG. 2) with a single mode fiber. For example, the short detector 1514a may be coupled to a prism 212 with a single mode fiber and the long detector 1514b may be coupled to a prism 212 with 7 single mode fibers. Fibers used together with detectors 1514a, 1514b may have about a 50 μm diameter. In aspects of the present disclosure the fiber used for the source 1512 may have a 62.5 μm core and/or 125 μm cladding. The fiber used for the detector may have a 9 μm core and/or 125 μm cladding.

Fibers may be glued into ferrules 220, 222, 706, 808 (see FIGS. 2, 7, and 8) using an adhesive and the ends polished prior to use in the probe. For example, an optical fiber polisher such as GT-2000A (as sold by Agate Code Technology Corp, headquartered in the U.S. under the company name RubeNova Inc., Stockton, Calif.) with diamond paper then cleaned in ultrasonic cleaner. For a long detector 1514b, a ferrule 222 combing multiple fibers may be used. Ferrules may be centered on a prism, for example with about 0.25 mm from the center of about a 3.5 mm by about 3.5 mm prism. Air bubbles may form in the adhesive when forming ferrules, particularly between the fiber tip and a prism and should be carefully avoided as they may reduce transmission efficiency. Two fibers may also be spiced together. Fibers may be straight or may feature a degree turn, for example a 90-degree turn.

Various connectors may be used together with fibers, for example an MTP® connector, as sold by US Conec Ltd., Hickory, N.C. The connector may be used to connect the fiber 1508, 1510 to a controller, e.g. the laptop 1202, that allows user input to systems of the present disclosure. Fiber length to the controller may be varied depending on the need for varying controller positions. For example, if the controller is on a table or card adjacent to the patient, a 3 m long fiber may be used. Extender cables may be used to extend the fiber as needed.

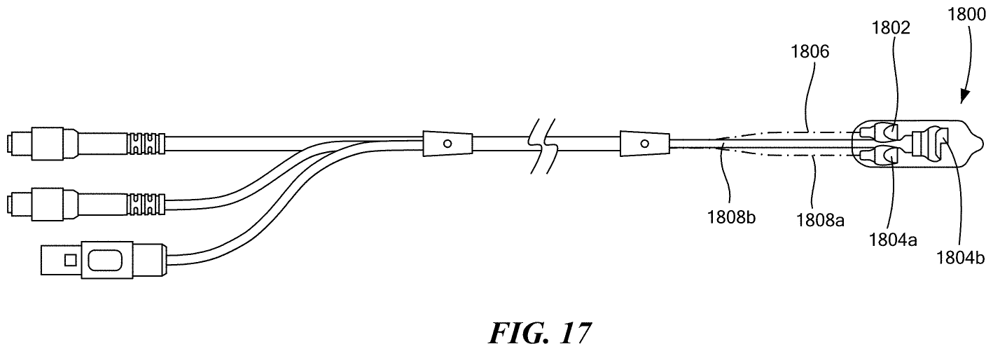

FIG. 17 depicts various fibers and connectors that may be used in connection with aspects of the present disclosure. For example, a probe 1800 can include a single source 1802, a short detector 1804a, and a long detector 1804b. A multi-mode optical fiber 1806 can connect the single source 1802 to a controller, while a single-mode optical fiber 1808a, 1808b can be used to couple the short detector 1804a and long detector 1804b, respectively, to the controller.

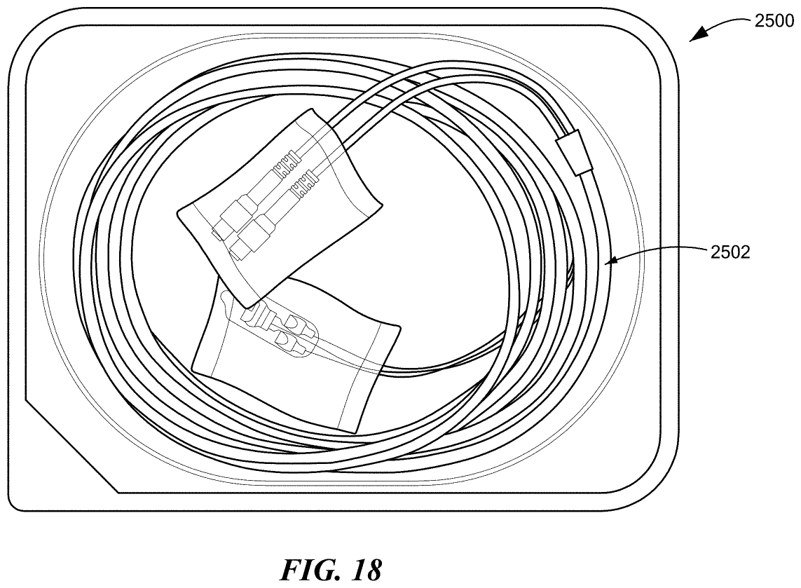

FIG. 18 shows packaging 2500 for fiber cables 2502, which can include, for example, a combination of multi-mode and single-mode optical fibers as illustrated in FIG. 17, that can advantageously be used to protect and transport fiber cables.

Methods of the present disclosure may be performed by using any of the systems of the present disclosure as described herein or derivable therefrom. Methods of the present disclosure for monitoring contact between a probe and a subject can include contacting a probe to a subject, transmitting a first electromagnetic radiation signal from a source in the probe to the subject, detecting via at least one detector in the probe a second electromagnetic radiation signal from the subject, detecting via a processor coupled to the at least one detector a change in contact between the probe and the subject based on a change in the second electromagnetic radiation signal, and stopping via the processor the source from transmitting the first electromagnetic signal upon detecting the change in contact between the probe and the subject.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The devices, systems, and methods disclosed herein may be embodied in other specific forms without departing from the spirit or essential characteristics thereof. The embodiments described above are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A system for monitoring contact between a probe and a subject, the system comprising:
a probe configured to contact a subject, the probe including:
a source configured to transmit a first electromagnetic radiation signal to the subject; and
at least one detector configured to detect a second electromagnetic radiation signal from the subject;
a processor coupled to the probe, and
non-transitory memory containing instructions executable by the processor to cause the system to:
detect a change in contact between the probe and the subject based on a change in the second electromagnetic radiation signal; and
stop the source from transmitting the first electromagnetic signal.

2. The system of claim 1, wherein the subject is a human subject.

3. The system of claim 1, wherein the source comprises a Near-Infrared Spectroscope (NIRS), cerebral oximeter, diffuse correlative spectroscope (DCS), cerebral oximeters, or Laser speckle contrast imaging (LSCI) device.

4. The system of claim 3, wherein the first electromagnetic signal is a near-infrared signal.

5. The system of claim 4, wherein the change in the second electromagnetic radiation signal is selected from the group consisting of a change in intensity, a change in wavelength, and a Doppler shift.

6. The system of claim 1, wherein the processor detects the change in contact in real-time.

7. The system of claim 6, further comprising a wearable article configured to house the probe and maintain contact between the probe and the subject.

8. The system of claim 7, wherein contact between the probe and the subject is contact between the probe and a tissue of the subject.

9. The system of claim 8, wherein the tissue of the subject is skin.

10. The system of claim 2, wherein the human subject is less than 25 weeks of age.

11. The system of claim 1, further comprising at least one of an accelerometer or temperature sensor, wherein the non-transitory memory further contains instructions executable by the processor to cause the system to: detect a change in contact between the probe and the subject based on a change detected by the accelerometer or temperature sensor and stop the source from transmitting the first electromagnetic signal.

12. A method for monitoring contact between a probe and a subject, the method comprising:
contacting a probe to a subject;
transmitting a first electromagnetic radiation signal from a source in the probe to the subject;
detecting via at least one detector in the probe a second electromagnetic radiation signal from the subject;
detecting via a processor coupled to the probe change in contact between the probe and the subject based on a change in the second electromagnetic radiation signal; and
stopping via the processor the source from transmitting the first electromagnetic signal upon detecting the change in contact between the probe and the subject.

13. The method of claim 12, wherein the subject is a human subject.

14. The method of claim 13, wherein the first electromagnetic signal is generated by a Near-Infrared Spectroscope (NIRS), diffuse correlative spectroscope (DCS), cerebral oximeters, or a Laser speckle contrast imaging (LSCI).

15. The method of claim 14, wherein the first electromagnetic signal is a near-infrared signal.

16. The method of claim 15, wherein the change detected in the second electromagnetic radiation signal is selected from the group consisting of a change in intensity, a change in wavelength, and a Doppler shift.

17. The method of claim 12, wherein a change in contact is detected in real-time.

18. The method of claim 17, wherein the probe comprises a wearable article that maintains contact between the probe and the subject.

19. The method of claim 18, wherein contact between the probe and the subject is contact between the probe and a tissue of the subject.

20. The method of claim 19, wherein the tissue of the subject is skin.

21. The method of claim 12, wherein the human subject is less than 25 weeks of age.

22. The method of claim 12, wherein the probe further comprises at least one of an accelerometer or temperature sensor, and wherein the method further comprises detecting via a processor coupled to the at least one accelerometer or temperature sensor a change in contact between the probe and the subject based on a change detected by the accelerometer or temperature sensor and stop the source from transmitting the first electromagnetic signal.

23. A system for monitoring contact between a probe and a subject, the system comprising:
a probe configured to contact a subject, the probe including:
a source configured to transmit a first electromagnetic radiation signal to the subject; and
at least one of an accelerometer or temperature sensor;
a processor coupled to probe, and
non-transitory memory containing instructions executable by the processor to cause the system to:
detect a change in contact between the probe and the subject based on a change detected by the accelerometer or temperature sensor; and
stop the source from transmitting the first electromagnetic signal.

24. The system of claim 21, wherein the system comprises both an accelerometer and a temperature sensor.

25. The system of claim 22, wherein the non-transitory memory contains instructions executable by the processor to cause the system to detect a change in contact between the probe and the subject based on a change detected by both the accelerometer and temperature sensor.

26. The system of claim 1, wherein the probe further comprises at least one detector configured to detect a second electromagnetic radiation signal from the subject, and wherein the non-transitory memory contains instructions executable by the processor to cause the system to:
detect a change in contact between the probe and the subject based on a change in the second electromagnetic radiation signal; and
stop the source from transmitting the first electromagnetic signal.
